(19) United States
(12) Patent Application Publication
(10) Pub. No.: US 2004/0219158 A1
(43) Pub. Date: Nov. 4, 2004

(54) Compositions and methods for diagnosis and therapy of medical conditions involving infection with pseudomonas bacteria

(75) Inventor: John L. Magnani, Gaithersburg, MD (US)

Correspondence Address: SEED INTELLECTUAL PROPERTY LAW GROUP PLLC, 701 FIFTH AVE, SUITE 6300, SEATTLE, WA, 98104-7092, US

(73) Assignee: GlycoMimetics, Inc., Rockville, MD

(21) Appl. No.: 10/742,631

(22) Filed: Dec. 19, 2003

Related U.S. Application Data
(60) Provisional application No. 60/435,958, filed on May 2, 2003.

(51) Int. Cl. A61K 39/00; A61K 39/38

(52) U.S. Cl. 424/184.1

(57) ABSTRACT

Compositions and methods are provided related to Pseudomonas bacteria. The compositions and methods may be used for diagnosis and therapy of medical conditions involving infection with Pseudomonas bacteria. Such infections include Pseudomonas aeruginosa in the lungs of patients with cystic fibrosis. A compound useful in the present methods may be linked to a therapeutic agent. Pseudomonas bacteria may be inhibited by blocking colonization, arresting growth or killing the bacteria.

### CROSS-REFERENCE TO RELATED APPLICATION

This application claims the benefit of U.S. Provisional Patent Application No. 60/435,958 filed Dec. 20, 2002, where this provisional application is incorporated herein by reference in its entirety.

### BACKGROUND OF THE INVENTION

1. Field of the Invention

The present invention relates generally to compounds, compositions and methods for the diagnosis and therapy of diseases in warm-blooded animals (e.g., in humans) involving infections with and colonization by Pseudomonas bacteria, including Pseudomonas aeruginosa in the lungs of patients with cystic fibrosis. The invention relates more particularly to the use of one or more compounds selective for binding Pseudomonas bacteria. These compounds are useful for diagnosis and/or therapeutic intervention of the colonization of Pseudomonas bacteria, or may be linked to an agent(s) to target and effectively arrest or kill Pseudomonas bacteria.

2. Description of the Related Art

Pseudomonas infections occur in a variety of medical conditions and can be life threatening. Pseudomonas is an opportunistic bacterium. Examples of individuals at risk include cystic fibrosis patients and burn patients. Cystic fibrosis is described below as a representative example of a medical condition which can involve infection with Pseudomonas bacteria.

Cystic Fibrosis (CF) is the most common lethal genetic disease among the Caucasian population. CF is caused by mutations in the gene encoding the cystic fibrosis transmembrane conductance regulator (CFTR), which acts as a chloride channel. The genetic mutations of CFTR which alter ion movements also affect the N-glycosylation of CFTR as well as other cell surface molecules. All of the exocrine glands of the patients are affected; however, the lungs are the primary site of morbidity and mortality. The general change in glycosylation results in an increase in Lewis fucosylation and a decrease in sialylation. The salivary and respiratory musins from CF patients also contain higher levels of Lewis type oligosaccharides including sialylated and sulfated Lewis x/a structures.

The major cause of morbidity and mortality in CF patients is chronic lung colonization by the bacterium, Pseudomonas aeruginosa, which results in pronounced lung infection with a robust neutrophilic inflammatory response leading to destruction of the lungs and death. Colonization by P. aeruginosa initiates by the binding of fimbrial and flagellar lectins on the bacteria to Lewis-type carbohydrate structures on the lung cell surfaces. These lectins, known as PA-IL and PA-IIL, bind these oligosaccharide structures with high affinity and represent a potential molecular target to block the first step of bacterial colonization. Patients that are never fully colonized by the bacteria maintain an excellent long-term prognosis. Due to the difficulties in the current approaches in the art for prevention of colonization in an individual by Pseudomonas bacteria, there is a need for improved compounds, compositions and methods.

### BRIEF SUMMARY OF THE INVENTION

Briefly stated, this invention provides compounds, compositions and methods for utilizing lectins expressed on Pseudomonas bacteria for the detection of Pseudomonas bacteria and the diagnosis and therapy of disease involving Pseudomonas bacteria, including human disease. For example, glycomimetics of the Lewis structures that have high affinity binding to the lectins on P. aeruginosa will have a beneficial therapeutic effect on CF patients. Furthermore, these glycomimetics may be conjugated, for example, with strong antibiotics to increase the efficacy and lower the dose, thereby avoiding well known deleterious side effects of these potent antibiotics. Given that these binding sites are crucial for the colonization and pathogenicity of the bacterium, mutations in this target to become resistant to this conjugate therapy should result in non-pathogenic forms of the bacteria.

One embodiment of the present invention provides a method of inhibiting Pseudomonas bacteria in a warm-blooded animal comprising administering to the animal in an amount effective to inhibit the bacteria a compound comprising a compound according to FIG. 1 or FIG. 2.

In another embodiment, the present invention provides a conjugate comprising a therapeutic agent linked to a compound according to FIG. 1 or FIG. 2.

In another embodiment, the present invention provides a method of detecting Pseudomonas bacteria comprising contacting a sample with a diagnostic agent linked to a compound comprising a compound according to FIG. 1 or FIG. 2, under conditions sufficient for the compound to bind to the bacteria if present in the sample; and detecting the agent present in the sample, wherein the presence of agent in the sample is indicative of the presence of Pseudomonas bacteria.

In another embodiment, the present invention provides a method of immobilizing Pseudomonas bacteria on a solid support comprising contacting, under conditions sufficient for binding, a sample containing Pseudomonas bacteria with a compound comprising a compound according to FIG. 1 or FIG. 2 that is immobilized on a solid support; and separating the sample from the solid support.

In other embodiments, the compounds and conjugates described herein may be used in the preparation of a medicament for the inhibition of Pseudomonas bacteria.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIGS. 1A-1E show the structures of glycomimetic compounds.

FIGS. 2A-2B show the structures of additional glycomimetic compounds.

FIG. 3 depicts the synthesis of representative glycomimetic compound 3.

FIG. 4 depicts the synthesis of glycomimetic compound 21.

FIG. 5 depicts the synthesis of representative glycomimetic compound 15.

FIG. 6 depicts the acylation of intermediate 21 to give a variety of representative glycomimetic compounds.

FIG. 7 depicts the synthesis of compound 22.

FIGS. 8A-8B depict the synthesis of compounds 4, 6, 7, 8, 10, 12, 13, 14, 16, 17, 18 and 19.

FIG. 9 depicts the synthesis of compounds 22 and 23.

FIG. 10 depicts the synthesis of intermediates.

FIG. 11 depicts the synthesis of intermediates and compound 24.

FIG. 12 depicts the synthesis of intermediates xxxxiv and xxxxv.

FIG. 13 depicts the synthesis of compound 25.

FIG. 14 depicts the synthesis of compound 26.

FIG. 15 depicts the synthesis of intermediates xxxxxi and xxxxxii.

FIG. 16 depicts the synthesis of compound 27.

FIG. 17 depicts the synthesis of compound 28.

FIG. 18 depicts the binding of PA-IIL lectin to immobilized neutral carbohydrate structures.

FIG. 19 depicts the binding of PA-IIL lectin to immobilized acidic carbohydrate structures.

FIG. 20 depicts the determination of assay conditions for ICSO values of PA-IIL lectin inhibition.

FIG. 21 shows a schematic representation of the assay developed for determining IC₅₀ values of glycomimetic inhibitors of PA-IIL lectin.

FIG. 22 depicts the inhibition of PA-IIL lectin by glycomimetic compound 23.

### DETAILED DESCRIPTION OF THE INVENTION

As noted above, the present invention provides compounds and compositions that bind P. aeruginosa and may be used in the diagnosis and therapy of disease.

Glycomimetic Compounds

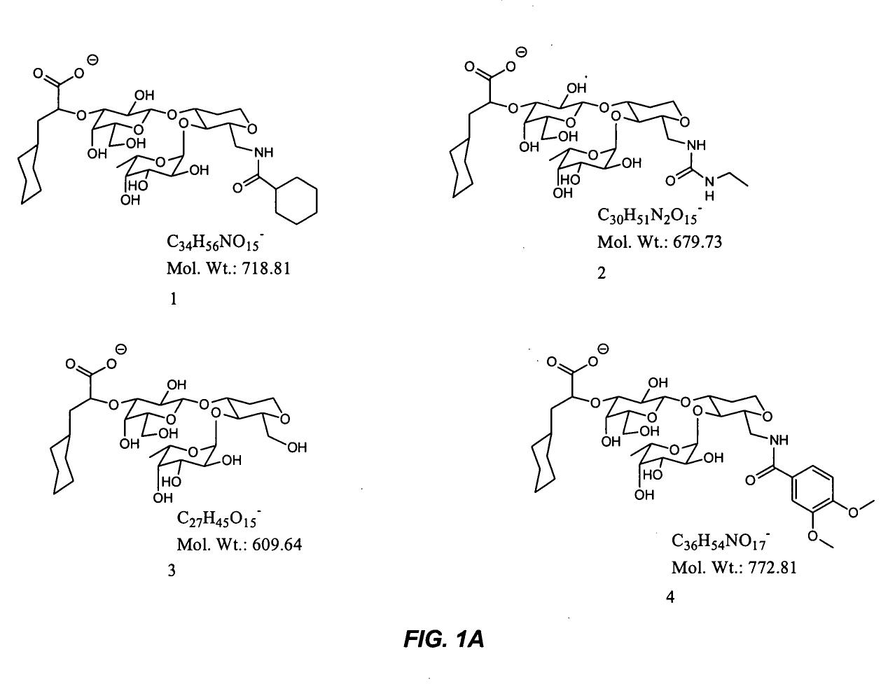

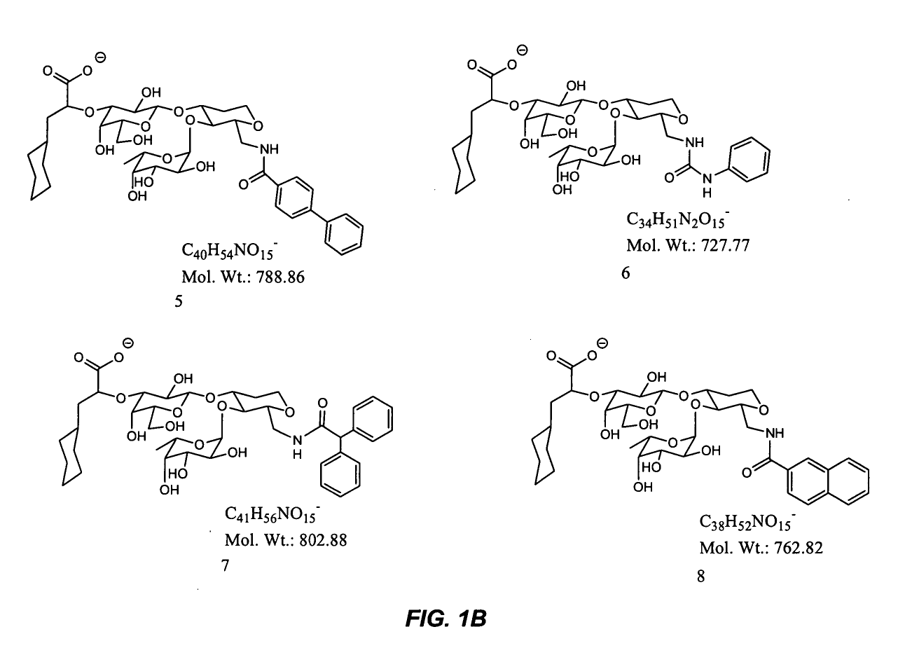

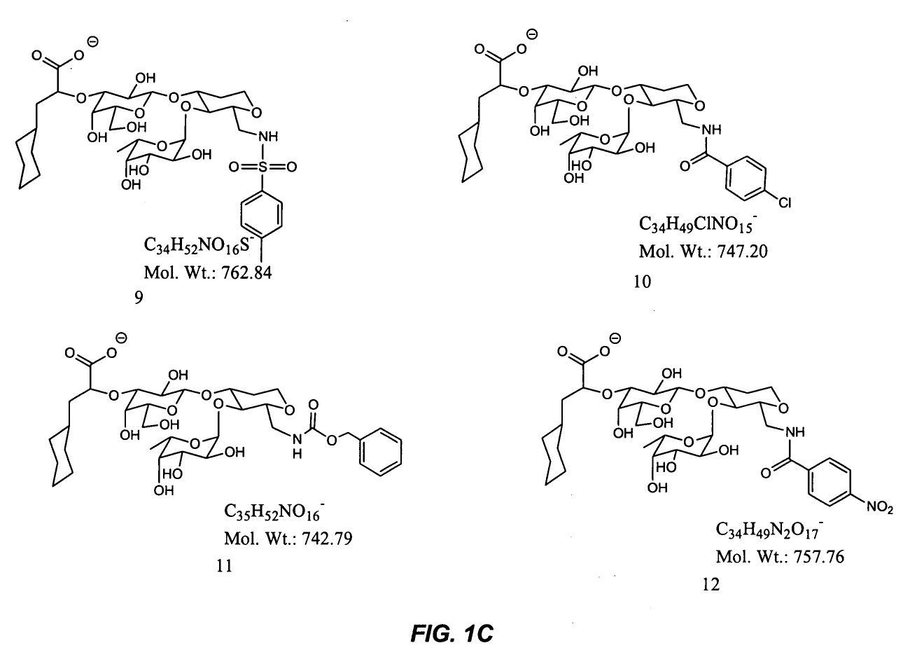

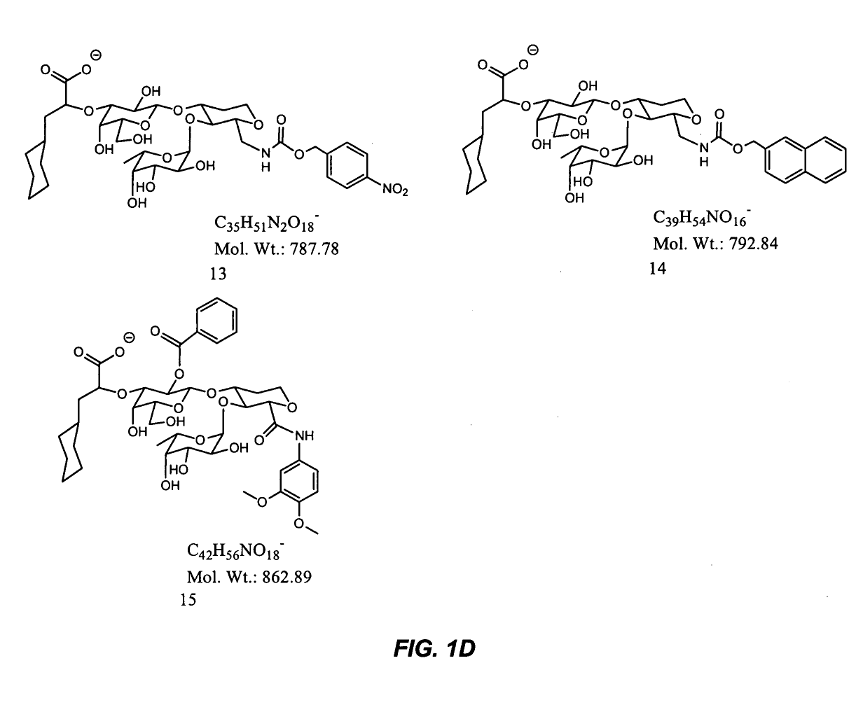

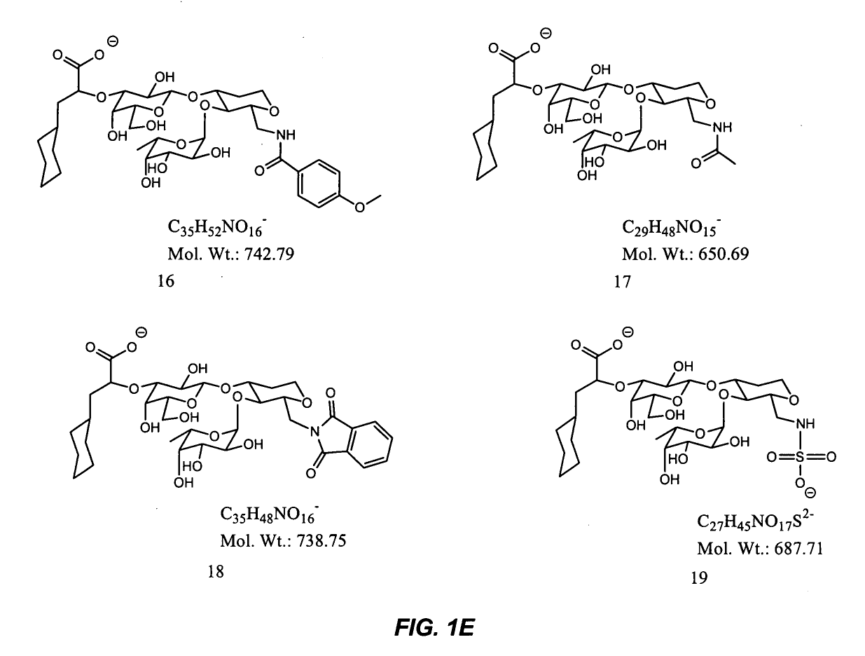

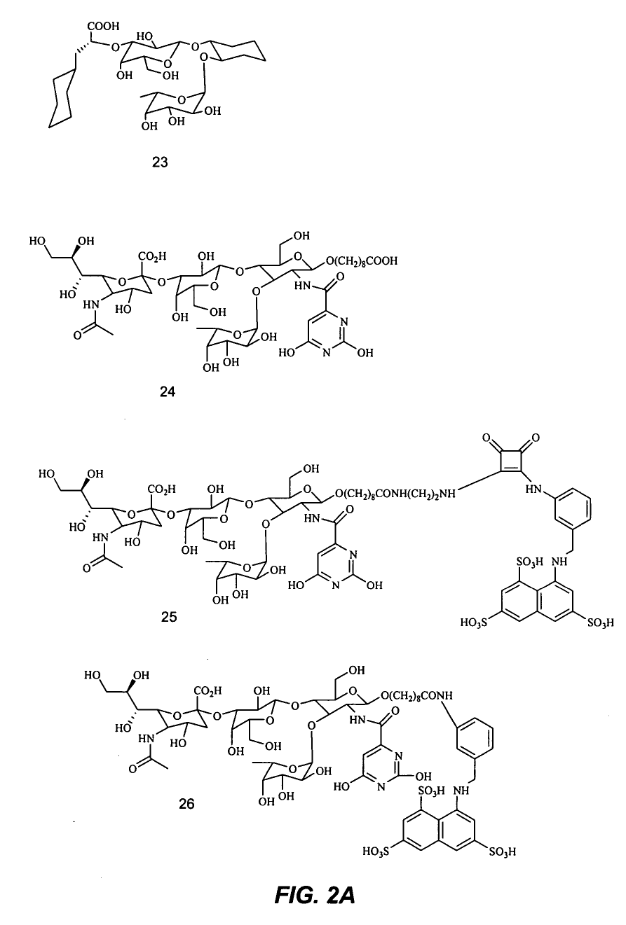

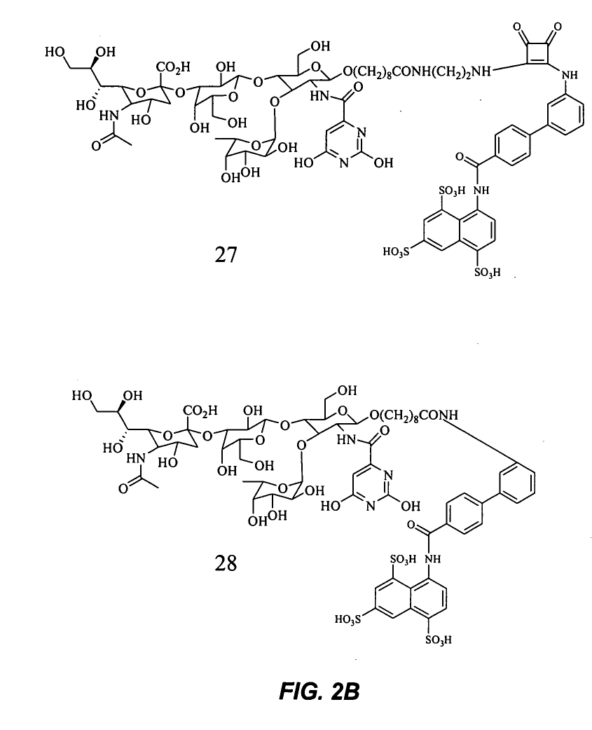

The term “Glycomimetic compound,” as used herein, refers to a molecule that binds specifically to P. aeruginosa. The structures of the Glycomimetic compounds covered by this invention are shown in FIGS. 1 and 2, and also include the compounds disclosed herein except that the compounds do not contain the mimic for sialic acid which is shown as the terminal cyclohexyl lactic acid moiety. This is accomplished, for example, by eliminating the step involving the addition of intermediate E in certain of the reaction schemes. All compounds (or conjugates thereof) useful in the present invention include physiologically acceptable salts thereof.

For certain embodiments, it may be beneficial to also, or alternatively, link a diagnostic or therapeutic agent, such as a drug to a Glycomimetic compound, to form a conjugate where the linkage is covalent. As used herein, the term “therapeutic agent” refers to any bioactive agent intended for administration to a warm-blooded animal (e.g., a mammal such as a human) to prevent or treat a disease or other undesirable condition or to enhance the success of therapies. Therapeutic agents include antibiotics, hormones, growth factors, proteins, peptides, genes, non-viral vectors and other compounds.

Glycomimetic Compound Formulations

Glycomimetic compounds as described herein may be present within a pharmaceutical composition. A pharmaceutical composition comprises one or more Glycomimetic compounds in combination with one or more pharmaceutically or physiologically acceptable carriers, diluents or excipients. Such compositions may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Within yet other embodiments, compositions of the present invention may be formulated as a lyophilizate. Compositions of the present invention may be formulated for any appropriate manner of administration, including for example, aerosol, topical, oral, nasal, intravenous, intracranial, intraperitoneal, subcutaneous, or intramuscular administration.

A pharmaceutical composition may also, or alternatively, contain one or more active agents, such as drugs, which may be linked to a Glycomimetic compound or may be free within the composition. The attachment of an agent to a Glycomimetic compound may be covalent or noncovalent.

The compositions described herein may be administered as part of a sustained release formulation (i.e., a formulation such as a capsule or sponge that effects a slow release of modulating agent following administration). Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of modulating agent release. The amount of Glycomimetic compound contained within a sustained release formulation depends upon the site of implantation, the rate and expected duration of release and the nature of the condition to be treated or prevented.

Glycomimetic compounds are generally present within a pharmaceutical composition in a therapeutically effective amount. A therapeutically effective amount is an amount that results in a discernible patient benefit, such as a measured or observed response of a condition associated with Pseudomonas infection.

Glycomimetic Compounds Methods of Use

In general, Glycomimetic compounds described herein may be used for achieving diagnostic and/or therapeutic results in disease (e.g., human disease) involving infection by Pseudomonas (e.g., P. aeruginosa) bacteria. Such diagnostic and/or therapeutic results may be achieved in vitro and/or in vivo in an animal, preferably in a mammal such as a human, provided that Pseudomonas (e.g., P. aeruginosa) is ultimately contacted with a Glycomimetic compound, in an amount and for a time sufficient to achieve a discernable diagnostic or therapeutic result. In the context of this invention, a therapeutic result would relate, for example, to the prevention of lung infections. In some conditions, therapeutic results would be associated with the inhibiting of Pseudomonas (including, for example, arresting the growth of or killing the bacteria or preventing colonization by the bacteria), such as P. aeruginosa. As used herein, therapy or therapeutic results includes treatment or prevention.

Glycomimetic compounds of the present invention may be administered in a manner appropriate to the disease to be treated or prevented. Appropriate dosages and a suitable duration and frequency of administration may be determined by such factors as the condition of the patient, the type and severity of the patient's disease and the method of administration. In general, an appropriate dosage and treatment regimen provides the modulating agent(s) in an amount sufficient to provide treatment and/or prophylactic benefit. Within particularly preferred embodiments of the invention, a Glycomimetic compound may be administered at a dosage ranging from 0.001 to 1000 mg/kg body weight (more typically 0.01 to 1000 mg/kg), on a regimen of single or multiple daily doses. Appropriate dosages may generally be determined using experimental models and/or clinical trials. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

Glycomimetic compounds may also be used to target substances to Pseudomonas bacteria, e.g., P. aeruginosa. Such substances include therapeutic agents and diagnostic agents. Therapeutic agents may be a molecule, virus, viral component, cell, cell component or any other substance that can be demonstrated to modify the properties of a target cell so as to provide a benefit for treating or preventing a disorder or regulating the physiology of a patient. A therapeutic agent may also be a prodrug that generates an agent having a biological activity in vivo. Molecules that may be therapeutic agents may be, for example, polypeptides, amino acids, nucleic acids, polynucleotides, steroids, polysaccharides or inorganic compounds. Such molecules may function in any of a variety of ways, including as enzymes, enzyme inhibitors, hormones, receptors, antisense oligonucleotides, catalytic polynucleotides, anti-viral agents, anti-tumor agents, anti-bacterial agents, immunomodulating agents and cytotoxic agents (e.g., radionuclides such as iodine, bromine, lead, rhenium, homium, palladium or copper). Diagnostic agents include imaging agents such as metals and radioactive agents (e.g., gallium, technetium, indium, strontium, iodine, barium, bromine and phosphorus-containing compounds), contrast agents, dyes (e.g., fluorescent dyes and chromophores) and enzymes that catalyze a colorimetric or fluorometric reaction. In general, therapeutic and diagnostic agents may be attached to a Glycomimetic compound using a variety of techniques such as those described above. For targeting purposes, a Glycomimetic compound may be administered to a patient as described herein.

Glycomimetic compounds may also be used in vitro, e.g., within a variety of well known cell culture and cell separation methods. For example, a Glycomimetic compound may be immobilized on a solid support (such as linked to the interior surface of a tissue culture plate or other cell culture support) for use in immobilizing Pseudomonas bacteria for screens, assays and growth in culture. Such linkage may be performed by any suitable technique, such as the methods described above, as well as other standard techniques. Glycomimetic compounds may also be used to facilitate cell identification and sorting in vitro, permitting the selection of such bacterial cells. Preferably, the Glycomimetic compound(s) for use in such methods is linked to a diagnostic agent which is a detectable marker. Suitable markers are well known in the art and include radionuclides, luminescent groups, fluorescent groups, enzymes, dyes, constant immunoglobulin domains and biotin. Within one preferred embodiment, a Glycomimetic compound linked to a fluorescent marker, such as fluorescein, is contacted with the cells, which are then analyzed by fluorescence activated cell sorting (FACS).

Such in vitro methods generally comprise contacting a sample (e.g., a biological preparation) with any one of the Glycomimetic compounds, and detecting the compound in the sample. If desired, one or more wash steps may be added to a method. For example, subsequent to contacting a sample with a Glycomimetic compound but prior to detection of the compound, the sample may be washed (i.e., contacted with a fluid and then removal of the fluid in order to remove unbound Glycomimetic compound). Alternatively, or in addition, a wash step may be added during the detection process. For example, if a Glycomimetic compound possesses a marker (a diagnostic agent) that can bind to a substance that is detectable, it may be desirable to wash the sample subsequent to contacting the sample with a detectable substance, but prior to the detection. As used herein, the phrase “detecting the compound (or agent) in the sample” includes detecting the compound (or agent) while it is bound to the sample, or detecting the compound (or agent) which was bound to the sample but after it has been separated from the sample.

The following Examples are offered by way of illustration and not by way of limitation.

### EXAMPLES

### Example 1

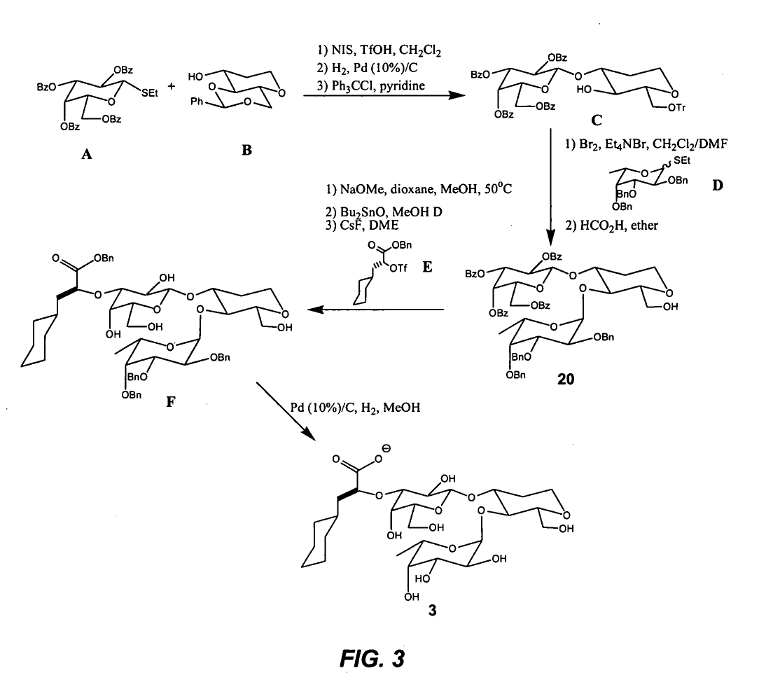

### Synthesis of 3 (FIG. 3)

Formation of Intermediate C:

Compounds A (5.00 g, 12.74 mmol) and B (4.50 g, 19.11 mmol) and NIS (3.58 g, 15.93 mmol) are dissolved in CH₂Cl₂ (50 ml) and cooled to 0° C. A solution of trifluoromethanesulfonic acid (0.15 M in CH₂Cl₂) is added dropwise with stirring. After the solution changes color from orange to dark brown, addition of TMS-OH ceases. The solution is then washed with saturated NaHCO₃ (30 ml) and the organic layer is dried with Na₂SO₄ and evaporated to dryness. The syrup obtained is purified by silica gel chromatography (hexane/ether, 1:1) and used in the next step.

The compound obtained previously is dissolved in THF (40 ml) and Pd (10%)/C (1/10 by mass) is added. The solution is degassed and an atmosphere of H₂ is generated. The reaction is allowed to proceed at RT until disappearance of starting material is confirmed by TLC. The solution is filtered thru a bed of celite and the filtrate is concentrated in vacuo giving the 4 and 6 OH compound. The compound is then dissolved in pyridine (25 ml) and cooled to 0° C. Ph₃CCl (1.2 eq) is added dropwise and the reaction is allowed to proceed at RT for 6 hrs. Ethyl acetate (50 ml) is then added and the solution is washed with 0.1 N HCl (2×50 ml), saturated NaHCO₃ (1×50 ml) and saturated NaCl (1×50 ml). The organic layer is dried with Na₂SO₄ and evaporated to dryness. Intermediate C is obtained by silica gel chromatography.

Formation of 20:

Compound C (800 mg, 1.41 mmol) and Et₄NBr (353 mg, 1.69 mmol) are dissolved in DMF/CH₂Cl₂ (10 ml, 1:1, containing molecular sieves) and cooled to 0° C. Br₂ (298 mg, 1.86 mmol, in CH₂Cl₂) is added dropwise to a separate solution of compound D (808 mg, 1.69 mmol) in CH₂Cl₂ at 0° C. After 30 min the Br₂/D solution is quenched with cyclohexene (0.2 ml) and added to the C solution immediately (within 10 min). This mixture is allowed to react for 65 hrs at RT. Ethyl acetate (100 ml) is added, the solution filtered, and the filtrate is washed with saturated NaS₂O₃ (2×50 ml) and saturated NaCl (2×50 ml). The organic layer is dried with Na₂SO₄ and evaporated to dryness. The resultant syrup is then dissolved in ether (50 ml) and formic acid (10 ml), is added with stirring. Upon completion of the reaction (as verified by TLC), the solution is washed with saturated NaHCO₃ (2×50 ml) and saturated NaCl (1×50 ml). The organic layer is then dried with Na₂SO₄, then evaporated to dryness. Compound 20 is then purified by silica gel chromatography.

Formation of Intermediate F:

Compound 20 (1 g, 1.02 mmol) is dissolved in MeOH/dioxane (10 ml, 20:1) and NaOMe (0.10 mmol) is added with stirring. The reaction is allowed to proceed at 50° C. for 20 hrs and then 2 drops of acetic acid are added. The solution is evaporated to dryness, dissolved in ethyl ether (25 ml) and washed with saturated NaCl (1×50 ml). The organic layer is dried with Na₂SO₄ and evaporated to dryness. The final product is purified by silica gel chromatography. The product (0.980 mmol) and Bu₂Sn (1.08 mmol) are suspended in MeOH (15 ml) and heated to reflux for 2 hrs. The resultant clear solution is then evaporated to dryness, taken up in pentane (10 ml) and evaporated giving a colorless foam. The foam is dissolved in 1,2-dimethoxyethane (DME, 15 ml), compound E (1.96 mmol) and CsF (1.18 mmol) are added and the reaction stirred for 2 hrs at room temperature. After 2 hrs, 1M KH₂PO₄ (50 ml) and KF (1 g) are added with stirring followed by extraction with ethyl acetate (2×25 ml). The organic layer is washed with 10% KF (2×50 ml) and saturated NaCl (2×50 ml), dried with Na₂SO₄ and evaporated to dryness under reduced pressure. Compound F is obtained via silica gel chromatography.

Formation of 3:

Compound F is dissolved in CH₃OH (50 ml) and Pd (10%)/C (1/10 by mass) is added. The solution is degassed and an atmosphere of H₂ is generated. The reaction is allowed to proceed at RT until disappearance of starting material is confirmed by TLC. The solution is filtered thru a bed of celite and the filtrate is concentrated in vacuo giving compound 3.

### Example 2

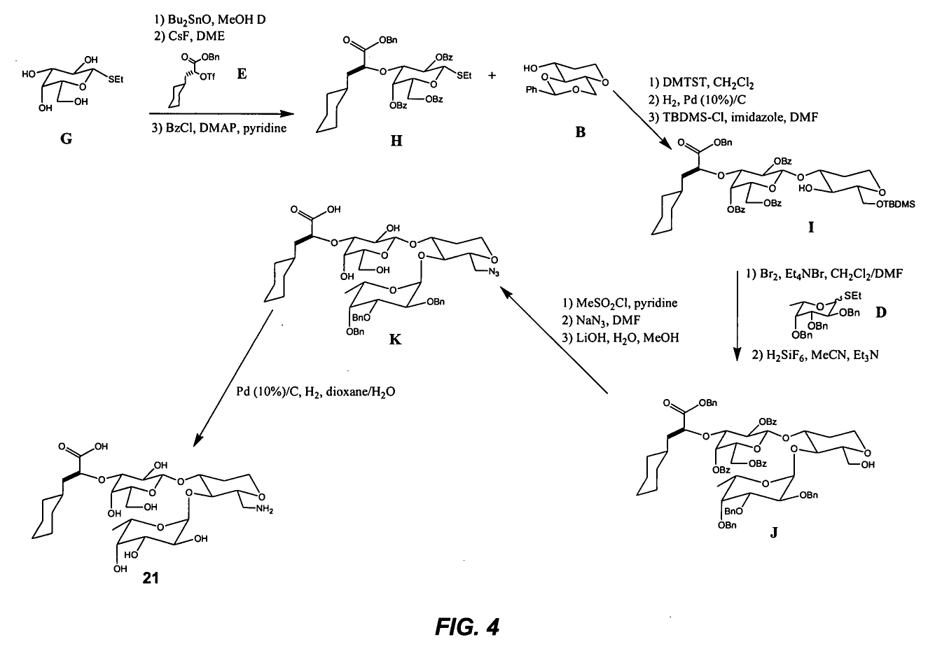

### Synthesis OF 21 (FIG. 4)

Formation of Intermediate H:

G (15.0 g, 66.9 mmol) and Bu₂SnO (20.0 g, 80.3 mmol) are suspended in MeOH (450 ml) and heated to reflux for 2 hrs. The resultant clear solution is then evaporated to dryness, taken up in pentane and evaporated again giving a colorless foam. The foam is dissolved in 1,2-dimethoxyethane (DME, 120 ml), E (39.6 g, 100.3 mmol) and CsF (12.2 g, 80.3 mmol) are added and the reaction stirred for 2 hrs at room temperature. After 2 hrs, 1M KH₂PO₄ (700 ml) and KF (25 g) are added with stirring followed by extraction with ethyl acetate (3×250 ml). The organic layer is washed with 10% KF (2×250 ml) and sat. NaCl (1×250 ml), dried with Na₂SO₄ and evaporated to dryness under reduced pressure. The compound (19.3 g, 41.2 mmol) is purified by silica gel chromatography and immediately dissolved in pyridine (210 ml) with a crystal DMAP. The solution is cooled to 0° C. and benzoyl chloride (52.1 g, 370.7 mmol) is added dropwise with stirring. The solution is allowed to warm to room temperature slowly and the reaction proceeds at RT for 20 min. The solution is evaporated to dryness, dissolved in ethyl acetate (500 ml), and washed with 0.1M HCl (2×250 ml), saturated NaHCO₃ (2×250 ml) and saturated NaCl (1×250 ml) solutions. The organic layer is dried with Na₂SO₄ and evaporated to dryness. H is obtained via silica gel chromatography.

Formation of Intermediate I:

H (10.0 g, 12.82 mmol) and B (6.05 g, 25.64 mmol) are dissolved in CH₂Cl₂ (75 ml) and 0.15M CF₃SO₃H (in CH₂Cl₂) is added dropwise at −10° C. with stirring. Addition is stopped when the orange solution changes to brown. Ethyl acetate (500 ml) is added and the solution is washed with sat NaHCO₃ (4×250 ml) and saturated NaCl (250 ml). The organic layer is then dried with Na_{2 SO}₄ and evaporated under reduced pressure. The compound (7.96 g, 9.19 mmol) is then purified by silica gel chromatography and then dissolved in DMF (55 ml). TBDMS-Cl (1.52 g, 10.1 mmol) and imidazole (0.94 g, 13.8 mmol) are then added and the reaction allowed to proceed at RT for 1 hr. Ethyl acetate (250 ml) is added and the solution washed with saturated NaHCO₃ (5×250 ml) and saturated NaCl (1×250 ml). The organic layer is then dried with Na₂SO₄ and purified by silica gel chromatography giving compound I.

Formation of Intermediate J:

Compound I (7.71 g, 7.87 mmol) and Et₄NBr (2.00 g, 9.45 mmol) are dissolved in DMF/CH₂Cl₂ (60 ml, 1:1, containing molecular sieves-12 g) and cooled to 0° C. Br₂ (1.90 g, 11.8 mmol) in CH₂Cl₂ (11 ml) is added dropwise to a separate solution of compound D (4.5 g, 9.45 mmol) in CH₂Cl₂ at 0° C. After 30 min, the Br₂/D solution is quenched with cyclohexene (2.5 ml) and added to the I solution immediately (within 10 min). This mixture is allowed to react for 65 hrs at RT. CH₂Cl₂ (250 ml) is added, the solution filtered, and the filtrate is washed with saturated NaHCO₃ (2×50 ml), 0.5M HCl (2×250 ml) and saturated NaCl (2×250 ml). The organic layer is dried with Na₂SO₄ and evaporated to dryness. The mixture is dissolved in MeCN (85 ml) at RT and a solution of Et₃N (0.21 ml) and H₂SiF₆ (1.3 ml, 35%) in MeCN (17 ml) is added and stirred for 2 hrs. CH₂Cl₂ (250 ml) is added and the solution washed with saturated NaHCO₃ (3×250 ml) and saturated NaCl (1×250 ml). The organic layer is dried with Na₂SO₄, evaporated to dryness and J is purified by silica gel chromatography.

Formation of Intermediate K:

J (12.5 g, 9.75 mmol) is dissolved in pyridine (80 ml) and methanesulfonylchloride (3.35 g, 29.2 mmol) is added dropwise with stirring over 5 min. The reaction is allowed to proceed for 30 min and then ethyl acetate (500 ml) is added. The solution is washed with 1N HCl (250 ml). The organic layer is dried with Na₂SO₄ and evaporated. The resultant syrup (12.95 g, 9.52 mmol) is dissolved in DMF (40 ml) and NaN₃ (4.64 g, 74.4 mmol) is added. The reaction is allowed to proceed for 35 hrs under argon atmosphere at 65° C. The solution is diluted with ethyl acetate (500 ml) and washed with H₂O (300 ml) and saturated NaCl (150 ml). The organic layer is dried with Na₂SO₃ and evaporated to dryness. The compound is purified by silica gel chromatography. The purified product (12.2 g, 9.33 mmol) is then suspended in MeOH/H₂O (200 ml/20 ml) solution and LiOH—H₂O (5.1 g, 121.3 mmol) was added. The reaction is allowed to proceed at 65° C. for 20 hrs. Ethyl ether (500 ml) was added and the solution is washed with saturated NaCl (200 ml). The organic layer is dried with Na₂SO₄ and evaporated to dryness. Compound K is purified via silica gel chromatography.

Formation of 21:

Compound K (8.45 g, 9.33 mmol) is dissolved in dioxane/H₂O (250 ml/50 ml) and Pd (10 %)/C (3.4 g) is added. The solution is degassed and an atmosphere of H₂ is generated. The reaction is allowed to proceed at RT until disappearance of starting material is confirmed by TLC. The solution is filtered thru a bed of celite and the filtrate is concentrated in vacuo giving compound 21.

### Example 3

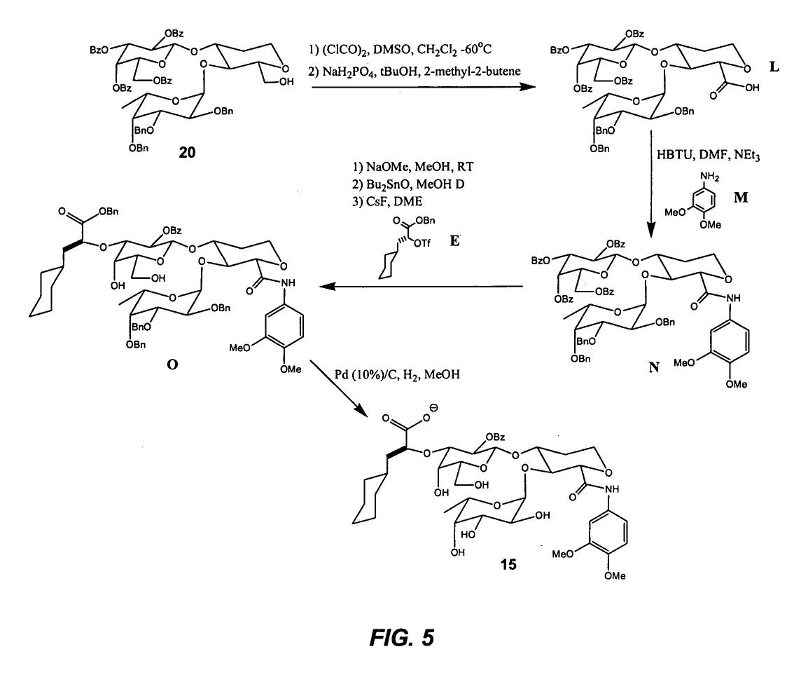

### Synthesis of 15 (FIG. 5)

Formation of Intermediate L:

Compound 20 (10 mmol) is dissolved in CH₂Cl₂ (30 ml) and DMSO (20 mmol) is added and the solution is cooled to −60° C. Oxalyl chloride (11 mmol) is added slowly to the stirred solution of 20. The reaction is allowed to proceed for 30 min under N₂ atmosphere. The reaction is washed with 0.1 M HCl, saturated NaHCO₃, and saturated NaCl. The organic layer is dried with Na₂SO₄ and evaporated to dryness. The resultant syrup is placed in tBuOH (20 ml) and 2-methyl-2-butene (10 ml) and NaH₂PO₄ (20 mmol) is added with stirring. The reaction is allowed to proceed for 3 hrs and is then evaporated taken up in CH₂Cl₂ and washed with 0.1 M HCl, saturated NaHCO₃, and saturated NaCl. The resultant compound is purified by silica gel chromatography giving compound L.

Formation of Intermediate N:

Compound L (10 mmol) is dissolved in DMF (15 ml) and compound M (10 mmol), HBTU (12 mmol) and Et₃N (20 mmol) are added with stirring. The reaction is allowed to proceed at RT for 24 hrs. Ethyl acetate (100 ml) is added and the solution is washed with 0.1 M HCl(1×100 ml), saturated NaHCO₃(1×100 ml), and saturated NaCl (1×100 ml). The organic layer is dried with Na₂SO₄ and evaporated to dryness. Compound N is isolated via silica gel chromatography.

Formation of Intermediate O:

Compound N (10 mmol) is dissolved in MeOH (35 ml) and NaOMe (1 mmol) is added with stirring. The reaction is allowed to proceed at RT for 20 hrs. The solution is evaporated to dryness, dissolved in ethyl ether (50 ml) and washed with saturated NaCl (1×50 ml). The organic layer is dried with Na₂SO₄ and evaporated to dryness. The final product is purified by silica gel chromatography. The product (0.980 mmol) and Bu_{2 Sn (}1.08 mmol) are suspended in MeOH (15 ml) and heated to reflux for 2 hrs. The resultant clear solution is then evaporated to dryness, taken up in pentane (10 ml) and evaporated giving a colorless foam. The foam is dissolved in 1,2-dimethoxyethane (DME, 15 ml), compound E (1.96 mmol) and CsF (1.18 mmol) are added and the reaction stirred for 2 hrs at room temperature. After 2 hrs, 1 M KH₂PO₄ (50 ml) and KF (1 g) are added with stirring followed by extraction with ethyl acetate (2×25 ml). The organic layer is washed with 10% KF (2×50 ml) and saturated NaCl (2×50 ml), dried with Na₂SO₄ and evaporated to dryness under reduced pressure. Compound 0 is obtained via silica gel chromatography.

Formation of 15:

Compound O (9 mmol) is dissolved in MeOH (200 ml) and Pd (10%)/C (3 g) is added. The solution is degassed and an atmosphere of H₂ is generated. The reaction is allowed to proceed at RT until disappearance of starting material is confirmed by TLC. The solution is filtered thru a bed of celite and the filtrate is concentrated in vacuo giving compound 15.

### Example 4

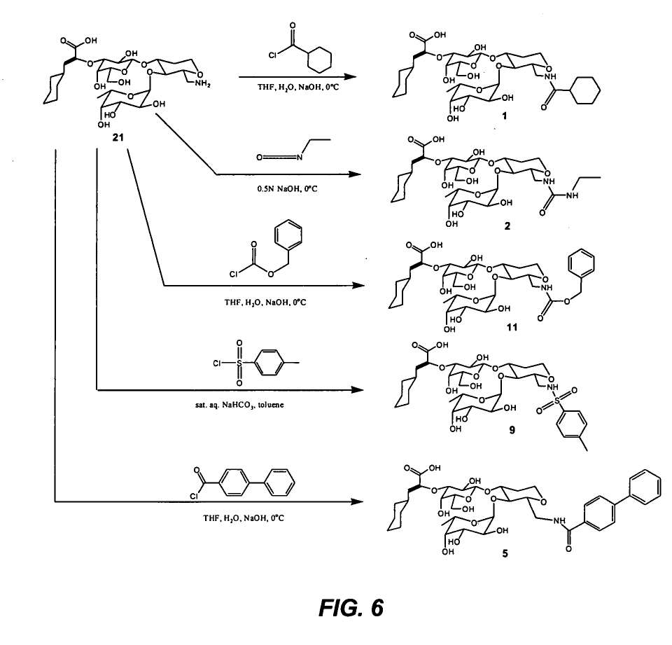

### Acylation of 21 (FIG. 6)

Reaction of 21 with Acid Chlorides:

Compound 21 (20 mg, 0.033 mmol) is dissolved in a THF/H₂O (2 ml, 1:1) solution containing 1N NaOH (pH adjusted between 8-10) and is cooled to 0° C. Cyclohexyl-carbonylchloride (0.049 mmol) is then added dropwise with stirring. The reaction is allowed to continue at 0° C. for 3 hrs. The solution is quenched with ice and the solution is evaporated to dryness. Compound 1 is purified by reverse phase chromatography.

Reaction of 21 with Isocyanates:

Compound 21 (30 mg, 0.049 mmol) is dissolved in a 0.5N aqueous NaOH solution (1 ml) and cooled to 0° C. Ethyl isocyanate (1.2 eq) is then added dropwise with stirring. The reaction is allowed to continue at RT for 3 hrs. The solution is quenched with ice and the solution is evaporated to dryness. Compound 2 is purified by reverse phase chromatography.

Reaction of 21 with Chloro-orthoformates:

Compound 21 (20 mg, 0.033 mmol) is dissolved in a THF/H₂O (2 ml, 1:1) solution containing NaOH (pH adjusted between 8-10) and is cooled to 0° C. Benzyl-chloro-orthoformate (0.049 mmol) is then added dropwise with stirring. The reaction is allowed to continue at 0° C. for 3 hrs. The solution is quenched with ice and the solution is evaporated to dryness. Compound 11 is purified by reverse phase chromatography.

Reaction of 21 with sulfonyl Chlorides:

Compound 21 (20 mg, 0.033 mmol) is dissolved in a saturated aqueous NaHCO₃/toluene (2 ml, 1:1) solution and is cooled to 0° C. p-Toluenesulfonyl chloride (0.049 mmol) is then added dropwise with stirring. The reaction is allowed to continue at 0° C. for 3 hrs. The solution is quenched with ice and the solution is evaporated to dryness. Compound 9 is purified by reverse phase chromatography.

### Example 5

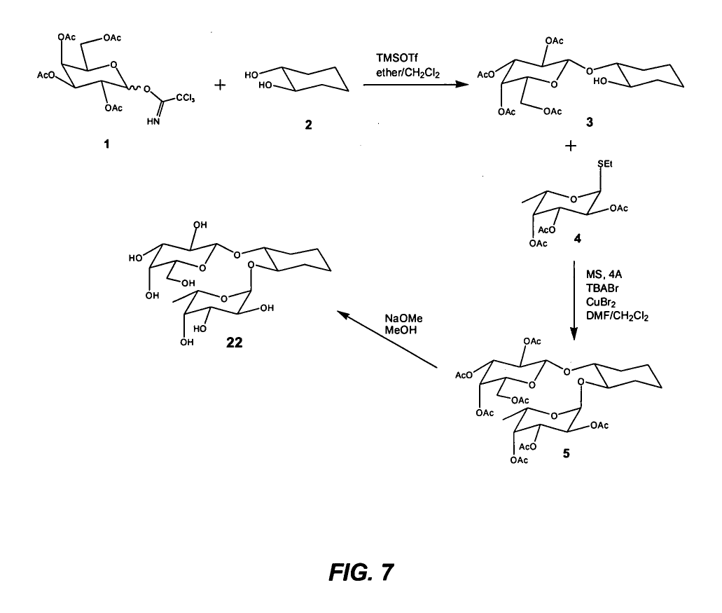

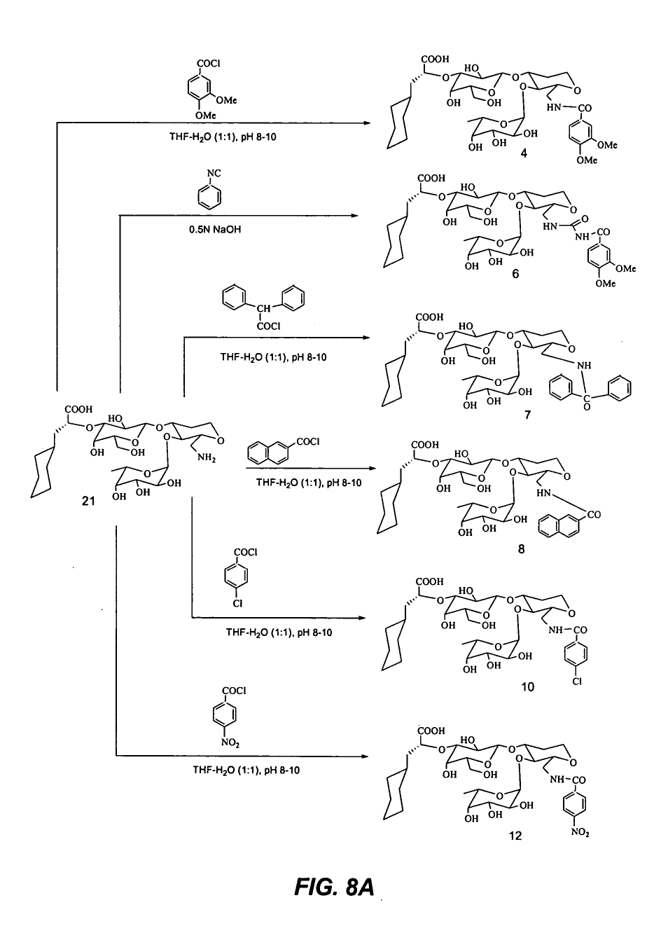

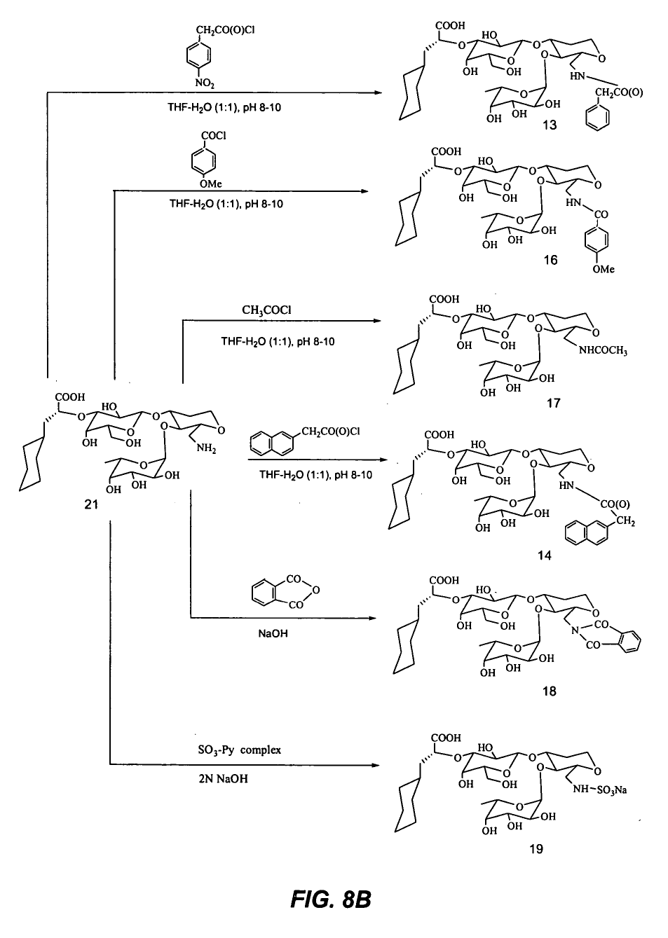

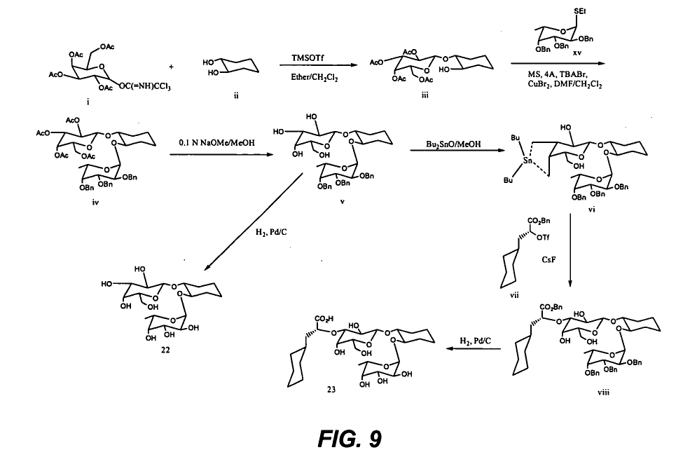

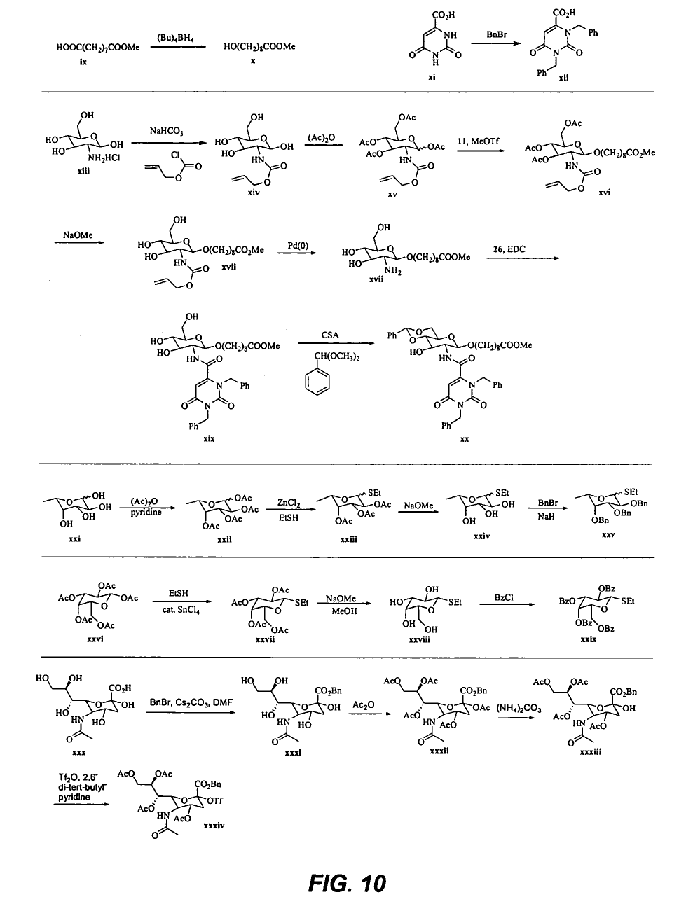

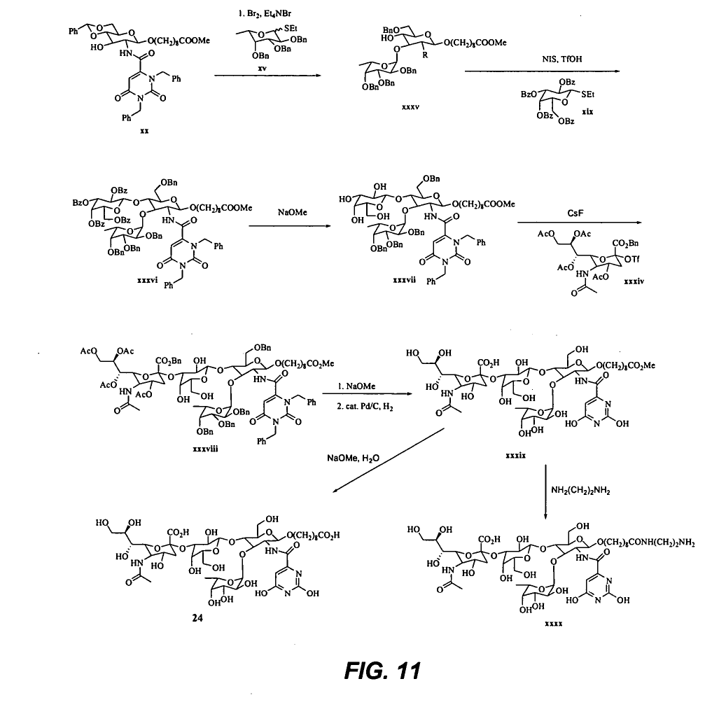

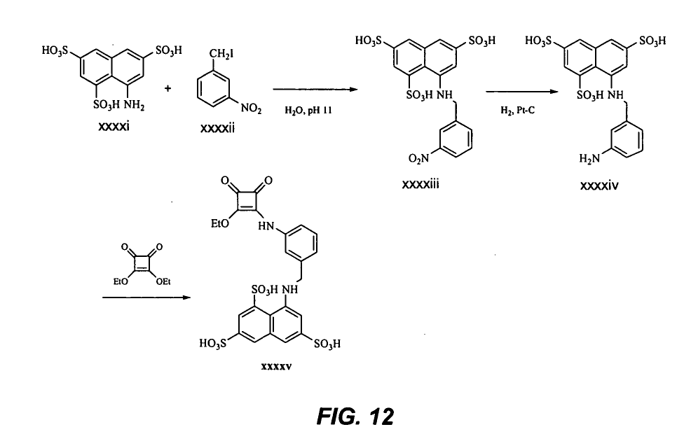

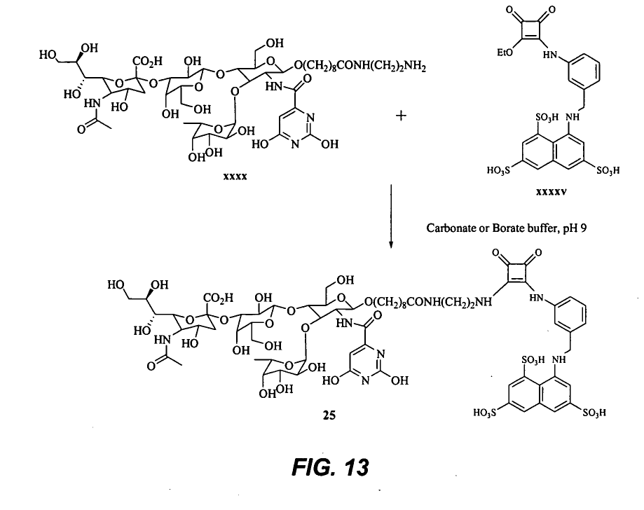

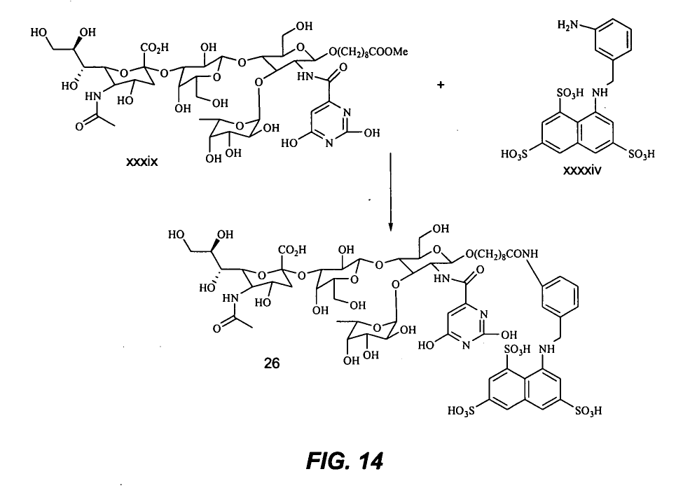

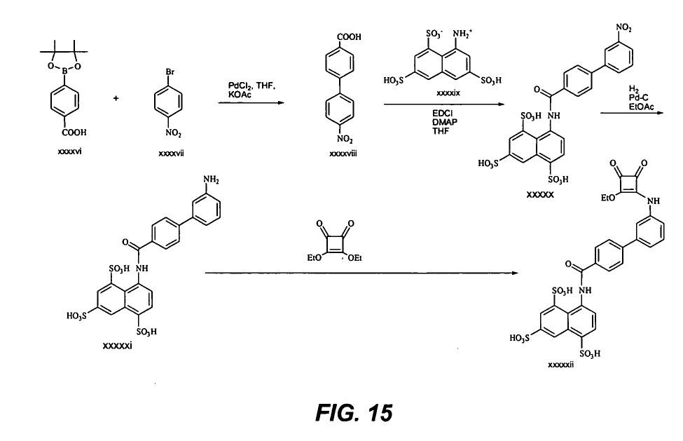

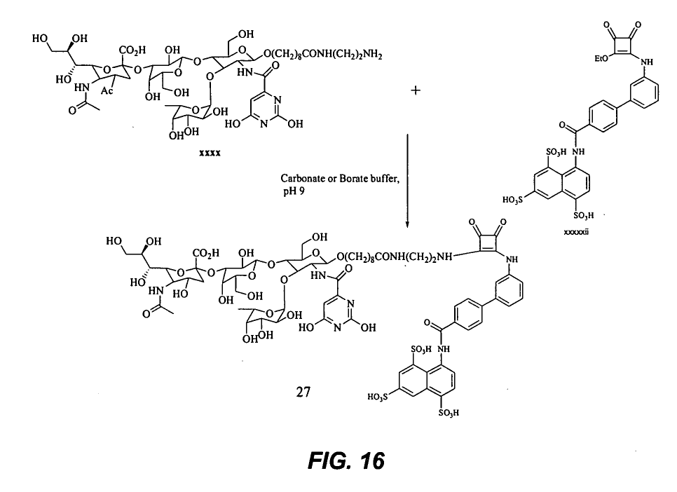

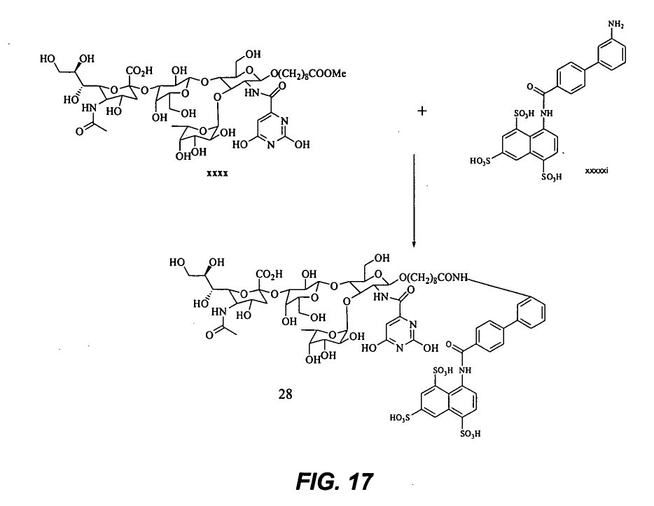

### Synthesis of Compound 22 (FIG. 7)

Compound 1 (2.77 mmol) is dissolved in a solution of diethyl ether/dichloromethane (1 ml, 1:1 mix) containing compound 2 (5.55 mmol). A 0.1 M TMSOTf (2.8 ml) solution is then added dropwise with stirring. After 30 min the solution is neutralized by addition of sodium bicarbonate (0.5 g), filtered and concentrated to dryness. Compound 3 is purified via column chromatography (toluene/acetone, 4:1 mix).

Compound 3 (1.96 mmol) is stirred in DMF/dichloromethane (50 ml, 1:5 mix) along with 4 A molecular sieves, TBABr (968 mmol) and CuBr₂ (529 mmol) for 1 hr. Compound 4 (2.94 mmol) is dissolved in dichloromethane (5 ml) and added dropwise to the solution. The solution is allowed to react for 24 hrs, filtered, and washed with saturated sodium bicarbonate and water (50 ml each). The organic layer is isolated, dried over sodium sulfate, and evaporated to dryness. Compound 5 is purified via column chromatography (hexane/ethyl acetate, 4:1 mix).

Compound 22 is obtained by dissolution of compound 5 in methanol followed by addition of 1 M sodium methoxide solution. The solution is allowed to react for 3-5 hrs and is then neutralized with Amberlite IR-120 resin, filtered and concentrated in vacuo. Compound 22 is purified by column chromatography (dichloromethane/methanol. 20:1 mix).

### Example 6

### Synthesis of Compounds 4, 6, 7, 8, 10, 12, 13, 14, 16, 17, 18, 19 AND 20

Starting from compound 21 (Example 2; FIG. 4) these compounds are synthesized by following the procedures described in the literature (Helvetica Chimica Acta Vol. 83, pp. 2893-2907, 2000; Angew Chem. Int Ed. Vol. 40, No. 19, pp. 3644-3647, 2001.)

### Example 7

### Synthesis of Compounds 23, 24, 25, 26, 27 and 28

Synthesis of intermediates of 23: Compound i (2.77 mmol) is dissolved in a solution of diethylether/dichloromethane (1 ml, 1:1 mix) containing compound 2 (5.55 mmol). A 0.1 M TMSOTf (2.8 ml) solution is then added dropwise with stirring. After 30 min the solution is neutralized by addition of sodium bicarbonate (0.5 g), filtered and concentrated to dryness. 3 is purified by silica gel chromatography to give compound iii.

Compound iii (1.96 mmol) is stirred in DMF/dichloromethane (50 ml, 1:5 mix) along with 4 A molecular sieves, TBABr (968 mmol) and CuBr₂ (529 mmol) for 1 hr. Compound xv (2.94 mmol) is dissolved in dichloromethane (5 ml) and added dropwise to the solution. The solution is allowed to react for 24 hr, filtered, and washed with saturated sodium bicarbonate and water (50 ml each). The organic layer is isolated, dried over sodium sulfate, and evaporated to dryness. iv is then purified by silica gel chromatography.

Compound iv is de-O-acetylated with 0.1N NaOMe in MeOH to give v and then treated with dibutyltin oxide in MeOH under refluxing condition to afford vi (after evaporation of the solvent) which is used for the next step without further purification.

Compound vi (1 mmol) is dissolved in DME (20 ml) and then added compound vii (2.5 mmol) and CsF (1.4 mmol). The resulting reaction mixture is stirred at room temperature for 8 hr, diluted with ethyl acetate and the organic layer is washed with water. The organic layer is concentrated to dryness and purified by silica gel chromatography to afford compound viii.

Synthesis of compound 23: Compound viii is hydrogenated in the presence of palladium in carbon to afford compound 23.

Synthesis of intermediate xx: Starting from N-acetyl glucosamine (5, 50 g) compound xx (50% overall yield) is synthesized following published procedures (Bioorg. Med. Chem. Lett. 111 pp. 923-925, 2001; Carbohydr. Res. 197, 75, 1990).

Synthesis of intermediate xv: Compound xv (15 g) is prepared from L-fucose following the procedure described in the literature (Carbohydr. Res. 201, 15-30, 1990).

Synthesis of intermediate xix: Compound xix is prepared from commercially available β-D-galactose-pentaacetate as described (WO 9701569; Chem. Astr., 126 186312, 1997).

Synthesis of intermediate xxxiv: Commercially available N-acetyl neuraminic acid (xxx, 10 g) is suspended in MeOH—H₂O (60 ml, 9:1) and the pH is adjusted to 8.1 by adding an aqueous solution of cesium carbonate. The solvent is removed and the residue is repeatedly evaporated with ethanol and then with hexane. The material is dissolved in DMF (65 ml) and benzyl bromide (3.5 ml) is added within 20 min. After the mixture is stirred for 16 hr, dichloromethane (100 ml) is added and washed with water (50 ml). The solvent is evaporated off and purified by silica gel chromatography to give xxxi in 68 % yield.

To a solution of compound xxxi (7 g) in pyridine (50 ml) is added acetic anhydride (48 ml) and the reaction mixture is stirred at RT for 16 hr. Solvent is evaporated off and the residue (xxxii) is dissolved in dry DMF (25 ml). To the mixture is added powdered ammonium carbonate (2 g) and the mixture is stirred for 12 hr at 28 degree centigrade. The mixture is added to a ice-cold solution of 1N HCl in water (50 ml) and dichloromethane (100 ml) is added. After solvent extraction, organic layer is evaporated off and then dried under vacuum for 24 hr. The residue is purified by silica gel chromatography to afford xxxiii in 71% yield.

Compound xxxiii is dissolved in dry dichloromethane and 2,6-di-tert-butyl-pyridine (5 g) is added. The solution is cooled down to −20 degree centigrade and trifluoromethanesulfonic anhydride (7 g) is added portionwise in 10 min. The mixture is stirred for 4 hr and diluted with dichloromethane (100 ml) and is added to a solution of potassium hydrogen phosphate (500 ml). The layers are separated and the organic layer is dried (sodium sulfate) and solvent is evaporated off to afford xxxiv which is used for the next step without further purification.

Synthesis of intermediate xxxv: To a mixture of compound xx (10 g) and compound xv (15 g) in dichloromethane (100 ml) is molecular sieves (4A, 8 g). After stirring at RT for 1 hr, tetraethylammonium bromide (5 g) is added. A solution of bromine (1 g) in dichloromethane (25 ml) is added dropwise during 1 hr. The reaction mixture is continued to stir for 3 hr, filtered through a bed of celite and washed successively with cold water, saturated solution of sodium bicarbonate and water. Solvent is evaporated off and subjected to silica gel chromatography.

The product was treated with sodium cyanoborohydride in THF and HCl in ether to afford compound xxxv in 70% overall yield after silica gel chromatography.

Synthesis of intermediate xxxvi: To a mixture of xxxv (10 g) and xix (7 g) in dichloromethane (80 ml) is added N-iodosuccinimide (15 g) and molecular sieves (4A, 8 g). The reaction mixture is placed in an ice bath. The solution is stirred at 0-5 degree for 30 min and a solution of triflic acid (0.2 ml) in dichloromethane (25 ml) is added dropwise during 1 hr with stirring. Stirring is continued for 2 hr, filtered through a bed of celite and washed successively with cold water, cold saturated solution of sodium bicarbonate and cold water. Solvent is evaporated off and purified by silica gel chromatography to afforded xxxvi in 68% yield.

Synthesis of intermediate xxxvii: Compound xxxvi (8 g) is treated with 0.05N NaOEt in MeOH (100 ml) for 4 hr and after neutralization with IR120 (hydrogen form) resin, the reaction mixture is filtered off. The solvent is evaporated off to afford compound xxxvii in 96% yield.

Synthesis of intermediate xxxviii: Compound xxxvii (5 g) is treated with dibutyltinoxide (1 g) in MeOH for 4 hr under reflux. The solvent is evaporated off and co-evaporated with toluene several times and finally the residue is dried under high vacuum for 24 hr.

The crude reaction mixture is dissolved in dimethoxyetahne (DME, 100 ml) and is added CsF (1.7 g), and compound xxxiv (2.5 g). The reaction mixture is stirred at RT for 8 hr and ethyl acetate (100 ml) is added. Organic layer is washed water and organic solvent is evaporated off. The product is purified by silica gel chromatography to afford xxxviii in 64% yield.

Synthesis of intermediate xxxix: Compound xxxviii (2 g) is de-O-acetylated with 0.01N NaOMe in MeOH (100 ml, 1 hr), the crude reaction mixture is neutralized with IR120 (hydrogen form) resin, and solvent is evaporated off.

Product from the above reaction is dissolved in dioxane-water (1:1, 50 ml) and 10% PD-C is added. The reaction mixture is stirred vigorously under hydrogen atmosphere for 22 hr, filtered through a bed of celite and the solvent is evaporated off. Silica gel chromatography of resulting syrup to afford xxxix in 77% yield.

Synthesis of compound 24: To a solution of compound xxxix in MeOH—H₂O is added a solution of NaOMe in MeOH and the reaction mixture is stirred for 2 hr at room temperature. Neutralization with IR 120 resin and evaporation to dryness affords compound 24.

Synthesis of xxxx: Compound xxxix is treated (500 mg) with ethylenediamine at 70 degree centigrade for 4 hr. Solvent is evaporated off and the syrupy residue is purified by silica gel chromatography to give compound xxxx in 77% yield.

Synthesis of compound xxxxv: 3-nitro-benzyl iodide is added to an aqueous solution (pH 11) of commercially available 8-aminonaphthalene-1,3,5-trisulfonic acid (xxxxxi) with stirring at room temperature. pH of the solution is adjusted to 1 and after evaporation of the solvent, the product xxxxiii is precipitated out from ethanol.

Platinum catalyzed hydrogenation of compound xxxxiii affords compound xxxxiv in 96% yield.

To a solution of compound xxxxiv in phosphate buffer (pH 7.1) is added commercially available squaric acid and the reaction mixture is stirred for 4 hr at RT. It is then purified by reverse phase HPLC to afford compound xxxxv.

Synthesis of compound 25: Compound xxxx (0.2 g) is dissolved in carbonate buffer (2 ml, pH 8.8) and compound xxxxv (0.4 g) is added. The reaction mixture is stirred at RT for 24 hr. Another batch (0.2 g) of compound xxxxv is added and stirring is continued for 20 h at RT. Solvent is evaporated off and the mixture is purified by reverse phase HPLC to afforded 25.

Synthesis of compound 26: Compound xxxix (0.1 g) is reacted with compound xxxxiv to afford compound 26 after purification by HPLC.

Synthesis of intermediate xxxxxii: Commercially available compound xxxxvi (4-(4,4,5,5-tetramethyl-[1,3,2]dioxaborolan-2-yl)-benzoic acid), 1 eq) and KOAc (3 equ) are placed in THF (25 ml). To the resulting slurry is added PdCl₂ and commercially available p-bromonitrobenzene (xxxxvii, 1.2 equ) with stirring and the mixture is gently heated to 80 degree centigrade. After 6 hr, the reaction mixture is evaporated to dryness, dissolved in dichloromethane (30 ml) and washed with distilled water and saturated solution of sodium bicarbonate. The resulting biphenyl compound xxxxviii is taken directly to the next step.

Compound xxxxviii, (1 eq), dimethylaminopyridine (catalytic amount, one crystal), and EDCl (1.05 eq) are dissolved in DMF or THF (20 ml) and allowed to react at RT for 10 min. Commercially available compound xxxxix (8-aminonaphthalene-1,3,5-trisulfonic acid) is added to the reaction mixture with stirring and the reaction is allowed to proceed at RT under nitrogen for 48 hr. The reaction mixture is then evaporated to dryness and purified by reverse phase chromatography to afforded compound xxxxx.

To a solution of compound xxxxx in EtOAc is added PD-C and the reaction mixture is stirred under an atmosphere hydrogen for 2 hr. The reaction mixture is filtered through a bed of celite and evaporated to dryness to afford compound xxxxxi.

To a solution of compound xxxxxi in phosphate buffer (pH 7.1) is added commercially available squaric acid and the reaction mixture is stirred for 4 hr at RT. It is then purified by reverse phase HPLC to afford compound xxxxxii.

Synthesis of compound 27: Compound xxxx (0.2 g) is dissolved in carbonate buffer (2 ml, pH 8.8) and compound xxxxxii (0.4 g) is added. The reaction mixture is stirred at RT for 24 hr. Another batch (0.2 g) of compound xxxxxii is added and stirring is continued for 20 h at RT. Solvent is evaporated off and the mixture is purified by reverse phase HPLC to afford 27.

Synthesis of compound 28: Compound xxxix is reacted with compound xxxxxi to afford compound 28 which is purified by HPLC.

Synthetic References:

The synthesis protocols for preparation of certain of the compounds included within this application are illustrated in the following references: Helvetica Chemica Acta Vol. 83, pp.2893-2907 (2000) and Angew. Chem. Int. Ed. Vol. 40, No. 19, pp. 3644-3647 (2001).

### Example 8

### Assays

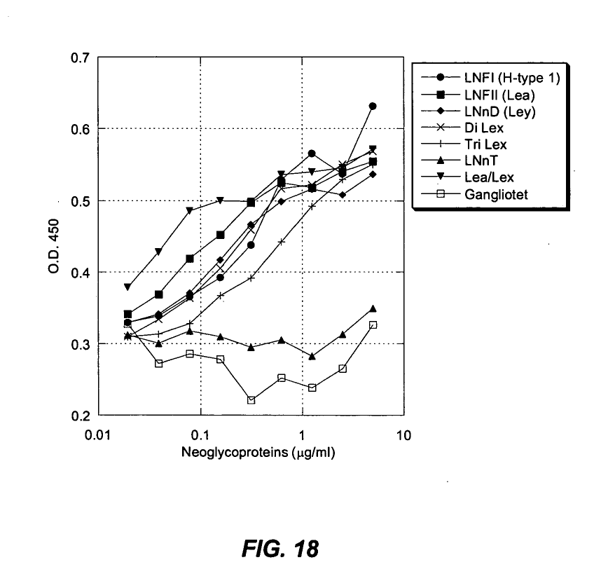

Purified oligosaccharides chemically coupled to albumin (neoglycoproteins) are coated in plastic microtiter wells. After blocking with BSA, the wells are then incubated and allowed to bind to purified PA-IIL lectin. Bound PA-IIL lectin is detected with anti-PA-IIL rabbit antisera followed by HRP-labeled anti-rabbit Ig and TMB reagent with color development by 1 M H₃PO₄ (FIG. 18). Structures and activities are presented in Table 1.

TABLE 1
Structures of immobilized neutral oligosaccharides screened for binding to
PA-IIL lectin.

| Carbohydrate Structure | Name | PA-IIL binding |
|---|---|---|
| Fucα1-2Galβ1-3GlcNAcβ1-3Galβ1-4Glc-R | LNFI(H-type 1) | + |
| 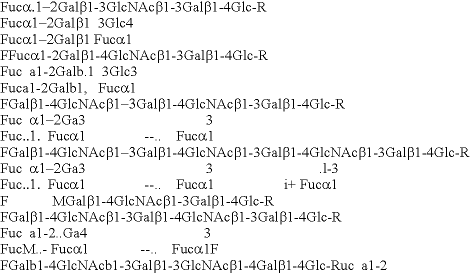 | Lea Ley DiLex TriLex LNnt Lea/Lex Gangliotetraose | |
| #+++++−+++− | | |

Results of screening immobilized neutral oligosaccharides are described in FIG. 18 and summarized below.
Type 1 lacto-series chains (e.g., Le^{a} structures) binding better to PA-IIL lectin than lacto-series type 2 chains (e.g., Le^{x} structures).
Extension of the type 1 structure as in Le^{a}/Le^{x} results in the most active compound in this series.

TABLE 2
Structures of immobilized acidic oligosaccharides screened for binding to
PA-IIL lectin

| | | PA-IIL |
|---|---|---|
| | | bind- |
| Carbohydrate Structure | Name | ing |
| 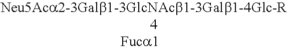 | SialylLea | ++ |
| 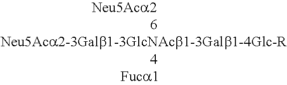 | DiSialylLea | + |
| 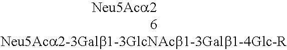 | DiSialylLNT | − |
| Neu5Acα2-3Galβ1-3GlcNAcβ1-3Galβ1-4Glc-R | LSTa | − |
| 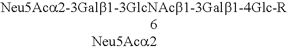 | LSTb | − |
| 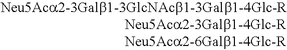 | LSTa 3′SL 6′SL | −−− |

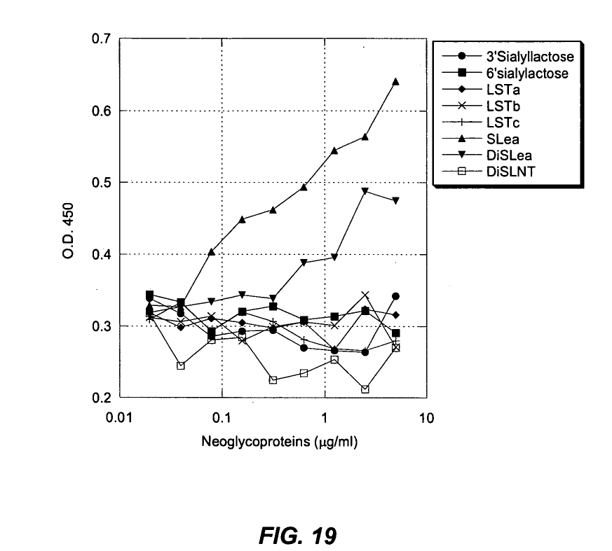

Results of screening immobilized acidic oligosaccharides are described in FIG. 19 and summarized below.
Only those sialylated oligosaccharides containing fucose will bind PA-IIL lectin.
The second sialyl group on disialyl Le^{a} appears to inhibit binding.

Purified oligosaccharides chemically coupled to albumin (neoglycoproteins) are coated in plastic microtiter wells. After blocking with BSA, the wells are then incubated and allowed to bind to purified PA-IIL lectin. Bound PA-IIL lectin is detected with anti-PA-IIL rabbit antisera followed by HRP-labeled anti-rabbit Ig and TMB reagent with color development by 1 M H₃PO₄ (FIG. 19). Structures and activities are presented in Table 2.

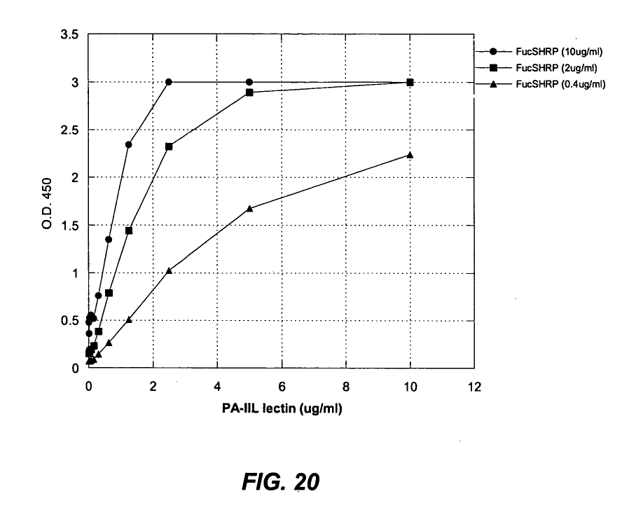

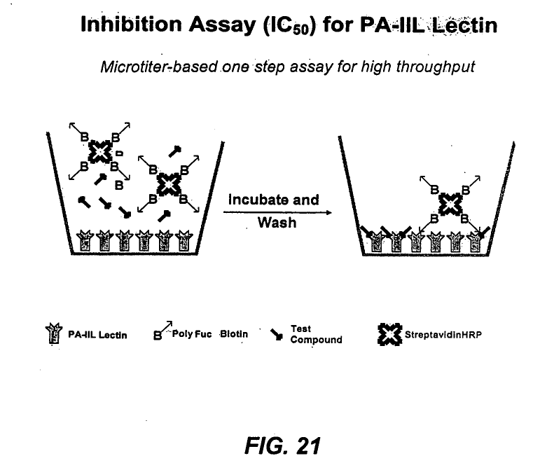

Biotinylated polymers of Fucose (Fuc-PAA-biotin, GlycoTech Corp.) are allowed to couple HRP-labeled streptavidin (KPL labs) overnight at 4° C. PA-IIL lectin is immobilized in plastic microtiter wells and allowed to react with the HRP-labeled fucosylated polymer. Binding is detected by TMB substrate followed by color development with 1 M H₃PO₄ (FIG. 20). Conditions chosen for the assay are coating with 3 μg/ml PA-IIL and incubation of inhibitor with 2 μg/ml of fucosylated polymer.

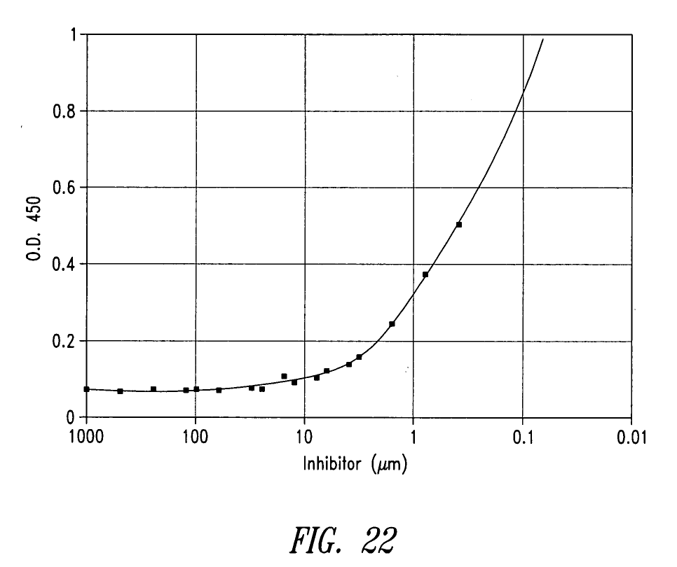

Compound 23 inhibited PA-IIL with an IC₅₀ of 420 nM (FIG. 22), while native saccharide inhibitor, Mannose, inhibited PA-IIL lectin with an IC₅₀ of 95 μM and the negative control saccharide, Galactose, showed no inhibition (data not shown).

From the foregoing it will be appreciated that, although specific embodiments of the invention have been described herein for purposes of illustration, various modifications may be made without deviating from the spirit and scope of the invention.

1. A method of inhibiting Pseudomonas bacteria in a warm-blooded animal comprising administering to the animal in an amount effective to inhibit the bacteria a compound comprising a compound according to FIG. 1 or FIG. 2.

2. The method of claim 1 wherein the compound is linked to a therapeutic agent.

3. The method of claim 1 or claim 2 wherein the compound is in combination with a pharmaceutically acceptable carrier or diluent.

4. The method of claim 1 wherein the bacteria are Pseudomonas aeruginosa.

5. A conjugate comprising a therapeutic agent linked to a compound according to FIG. 1 or FIG. 2.

6. The conjugate of claim 5 wherein the conjugate is in combination with a pharmaceutically acceptable carrier or diluent.

7. A method of detecting Pseudomonas bacteria comprising contacting a sample with a diagnostic agent linked to a compound comprising a compound according to FIG. 1 or FIG. 2, under conditions sufficient for the compound to bind to the bacteria if present in the sample; and detecting the agent present in the sample, wherein the presence of agent in the sample is indicative of the presence of Pseudomonas bacteria.

8. The method of claim 7 wherein the bacteria are Pseudomonas aeruginosa.

9. A method of immobilizing Pseudomonas bacteria on a solid support comprising contacting, under conditions sufficient for binding, a sample containing Pseudomonas bacteria with a compound comprising a compound according to FIG. 1 or FIG. 2 that is immobilized on a solid support; and separating the sample from the solid support.
